(19) United States
(12) Patent Application Publication
(10) Pub. No.: US 2023/0149303 A1
(43) Pub. Date: May 18, 2023

(54) BUCCAL AND ENTERIC DELIVERY OF FATTY ACIDS IN FOODS AND BEVERAGES

(71) Applicant: Douglas Thomas, Palo Alto, CA (US)

(72) Inventor: Douglas Thomas, Palo Alto, CA (US)

(21) Appl. No.: 17/906,946

(22) PCT Filed: Feb. 22, 2021
(86) PCT No.: PCT/US21/19135

§ 371 Date: Sep. 21, 2022

Related U.S. Application Data
(60) Provisional application No. 62/979,678, filed on Feb. 21, 2020.
(60) Provisional application No. 62/980,121, filed on Feb. 21, 2020.

(51) Int. Cl. A61K 9/00; A61K 31/05; A61K 9/48; A61K 31/522; A61K 31/4415; A61K 31/198

(52) U.S. Cl. CPC A61K 9/006; A61K 31/05; A61K 9/4858; A61K 31/522; A61K 31/4415; A61K 31/198

(57) ABSTRACT

A first technique for ameliorating bioactivity deterioration in fatty acid delivery includes retarding oxidation of fatty acids during manufacture, transportation, and storage of the fatty acid. A second technique for ameliorating bioactivity deterioration in fatty acid delivery includes enteric microencapsulation to avoid destruction of fatty acids in the stomach in favor of dissolving the fatty acids in the small intestines.

### CROSS REFERENCE TO RELATED APPLICATIONS

This application is a national phase application pursuant to 35 U.S.C. § 371 of International Application No. PCT/US2021/019135 filed on Feb. 22, 2021, which claims priority to U.S. Provisional Application No. 62/979,678 filed on Feb. 21, 2020 and U.S. Provisional Application No. 62/980,121 filed on Feb. 21, 2020, the disclosures of which are hereby incorporated by reference herein.

### BRIEF DESCRIPTION OF THE DRAWINGS

FIG. 1 is a diagram of an example of a controlled dosage dispenser.

### DETAILED DESCRIPTION

Fatty acid formulations have been found to be useful as liver enzyme inhibitors, anti-anxiety treatments, concentration/study aids, sleep aids, pan-peripheral pain management analgesics, inflammation reduces, and mechanisms for improving mood, to name several. A problem with delivery of fatty acids to consumers is in the deterioration of the fatty acids during manufacture, transportation, storage, and, after oral delivery, in the stomach. A first technique for ameliorating bioactivity deterioration in fatty acid delivery includes retarding oxidation of fatty acids during manufacture, transportation, and storage of the fatty acid. A second technique for ameliorating bioactivity deterioration in fatty acid delivery includes enteric microencapsulation to avoid destruction of fatty acids in the stomach in favor of dissolving the fatty acids in the small intestines.

An oral delivery system can include a bioactive component suspension. A suspension is a heterogeneous mixture in which the solute particles do not dissolve, but get suspended throughout the bulk of the solvent, left floating around freely in the medium. A suspension is typically defined as a heterogeneous mixture that contains solid particles sufficiently large for sedimentation, though the mixture is only classified as a suspension when and while the particles have not settled. Colloid suspensions are characterized as mixtures with particles (sometimes referred to as colloids) that do not settle or that would take a long time to settle appreciably. Colloids have a diameter of 1 micron or less (sometimes as small as 1 nanometer). Non-colloid solutions have suspended particles with a diameter of more than 1 micron (often as large as 50 microns in liquid without becoming noticeable on the palate or as large as 100 microns in food without becoming noticeable on the palate).

Suspensions are classified based on a dispersed phase and a dispersion medium. To create a suspension, typically an internal phase (solid) is dispersed throughout an external phase (fluid) through mechanical agitation, with the use of certain excipients or suspending agents. A suspension of liquid droplets or fine solid particles in a gas is called an aerosol (for non-colloid suspensions, it may be referred to as a spray or dust). Gases, liquids, and solids can be suspended in liquid to create a foam, emulsion, or liquid sol, which are the most applicable in this paper because each of these types of suspensions is represented by a number of common foods (e.g., whipped cream is a foam, mayonnaise is an emulsion, and milk (protein fraction) is a sol). Gases, liquids and solids can also be suspended in solids to create a solid foam, gel, or solid sol. Gels have the potential to deliver bioactives via a gelatin, such as a gummy bear. If it is desirable to draw a distinction between colloidal solutions and non-colloidal solutions, the term colloidal or non-colloidal can be appended to any of the terms aerosol, foam, emulsion, sol, or gel (e.g., colloidal aerosol or non-colloidal gel).

Fluids can include solutions; colloids and suspensions are different from solution, in which the dissolved substance (solute) does not exist as a solid, and solvent and solute are homogeneously mixed. Some examples of solutions with liquid dispersion mediums are oxygen (gas) dissolved in water, alcohol, and sugar (solid) dissolved in water.

An example of a suspension is enterically microencapsulated fatty acids in a fluid. The term “fluid” is sometimes used in such a manner as to be synonymous with “liquid”, but the term can have a broader definition, as was discussed in the preceding paragraphs. To the extent a distinction is desired, a fluid can be characterized as having a free surface in order to distinguish it from gaseous suspensions. A free surface is the surface of a fluid that is subject to zero parallel shear stress. Fluidized solids, including slurries, granular materials, and powders may form a free surface. Many foods are fluids, such as ketchup and syrup (pseudoplastic fluids), whipped cream and gummies (viscoelastic fluids), yogurt and peanut butter (thixotropic fluids), and custard (generalized Newtonian fluids), which makes delivery of bioactives via a fluid appealing.

In a specific implementation, a tincture has 40% to 60% alcohol as a suspension with polar and nonpolar aspects. In an alternative, the range is 10% to 80% for more concentrated/dilute bioactives. Although perhaps technically inaccurate, some people may refer to an “oil tincture” as a product that is between a tincture and an oil drop. In a specific implementation, an oil drop includes a hemp seed emulsion. In an alternative, the oil drop includes another oil, such jojoba, avocado, or olive oil, to name several. Short fatty acids can act as penetration enhancers (e.g., eucalyptol, limonene). In an alternative, bioactives are suspended in toilet water.

In a specific implementation a food-grade emulsifier is used (see the GRAS list) to create a product for consumption. A carrier oil can be used to dissolve gel-soluble components and water to dissolve water-soluble components. In the formulations provided in this paper, water is assumed to be the solvent unless otherwise explicitly stated or context dictates otherwise.

In a specific implementation, bioactives are enterically microencapsulated. Enteric microencapsulation is intended to represent enteric polymeric system added to the matrix of a dosage form or applied as a coating to produce a particle of between 1 and 100 microns with a bioactive that has a tractus digestorius delayed release. In a specific implementation, the bioactive is CBD that has a canalis gastrointestinales delayed release. Substances appropriate for such an implementation include shellac (esters of aleurtic acid), such as EmCoat 120 N and Marcoat 125; Zein; Cellulose Acetate Phthalate (CAP); cellulose acetate, such as Aquacoat CPD, Sepifilm L P, Klucel, Aquacoat ECD, and Metolose; Poly Vinyl Acetate Phthalate (PVAP), such as Sureteric; Hyroxylpropylcellulose; Hydroxy Propyl Methyl Cellulose (HPMC), such as HPMC 50, HPMC 55, HPMC 55-s; HydroxyPropyl MethylCellulose Phthalate (HPMCP); and acrylate polymers (methacrylate), such as Eudragit L and Eudragit S. In an alternative, the bioactive is THC, an omega 3 fatty acid, or some other fatty acid.

An example of a product that acts as a liver enzyme inhibitor will now be discussed. The specific implementation described can be characterized as a gummy candy formulation with:
50 mg CBD
0.5 mg pyridoxine tripalmitate
50 mg xylitol
5 mg caffeine
10 mg theanine

In this specific implementation, CBD is delivered via a gelatin candy (e.g., a gummy bear) with 50 mg CBD. It has been found that enzymes in the liver break down certain drugs but binding to CBD inhibits this liver function. The amount of CBD that must be delivered in this manner is relatively high, 5 to 20 mg/kg per day for the desired pharmacological effect. An example of a recommended dosage is one gummy bear (50 mg CBD) per 10 kg patient body weight per day, with the potential of allowing up to four times that amount to be consumed as needed. It would be desirable to make the dosage as precise as possible to ensure safety, reduce degradation of the product through oxidation during manufacturing, transportation, and storage, and to ensure enteric delivery of the CBD to avoid degradation of the CBD in the stomach.

B6 helps the body produce neurotransmitters (e.g., serotonin); it can block glutamate and help the brain learn to calm. As a point of comparison, B6 is typically about 75% bioavailable when consumed and absorbed tractus digestorius, while a mucosal quick release of pyridoxine tripalmitate, which is more effective at going across mucosal barriers than other forms of vitamin B6 is over 90% bioavailable if retained in the mouth for several seconds. B6 can also be delivered enterically with nearly 100% bioavailability, as well.

Pyridoxine tripalmitate dosage can be reduced from the amounts provided for this specific implementation but should exceed 0.1 mg to have pharmacological effect. As high as 50 mg pyridoxine tripalmitate could continue to see mood elevation effects, though beyond 50 mg the therapeutic advantage is not expected to increase. However, the recommended maximum daily intake of B6 is: Children 1-3 years, 30 mg; Children 4-8 years, 40 mg; Children 9-13 years, 60 mg; Adults, pregnant and breast-feeding women, 14-18 years, 80 mg; and Adults, pregnant and breast-feeding women, over 18 years, 100 mg. It would be undesirable for the product to cause a patient to exceed these amounts. While remaining safe, pyridoxine tripalmitate dosage can be increased from the amounts provided for this specific implementation but should not exceed about 5 mg.

It is relatively common for childhood epilepsy to be triggered by anxiety. Advantageously, it has been found CBD and B6 have an anti-anxiety effect. The CBD necessary for liver enzyme inhibition is far higher than the CBD needed, in conjunction with B6, to have an anti-anxiety effect. In the specific implementation described above, 25% (12.5 mg) of the CBD can be microencapsulated for buccal delivery. Due to a relatively high log P (XLogP3-AA 6.5), CBD is readily absorbed via mucosa. Other endocannabinoids also typically have relatively high log P. Xylitol, while providing a sweetener to the gummy bear, also acts as a penetration enhancer, which is useful because it reduces the amount of time a patient must chew to absorb a desired amount of the proportion of CBD intended for buccal delivery. Pyridoxine tripalmitate provides B6 in a form that is particularly suitable for absorption through mucosa due to its exceptionally high log P (e.g., XLogP3-AA 22.4).

In a specific formulation, CBD, xylitol, and pyridoxine tripalmitate are provided to a patient via buccal delivery when a gummy bear is chewed. Advantageously, the CBD and B6 are rapidly absorbed into the bloodstream to provide a rapid anti-anxiety effect. The remainder of the CBD is enterically delivered to ensure maximum bioavailability for the purpose of liver enzyme inhibition.

A low dose of CBD can act as a stimulant but, due to the high dosage necessary for liver enzyme inhibition, it is expected the CBD will act as a sedative when an entire dose of CBD (typically multiple gummy bears in this example) is absorbed. Indeed, the dosage is far higher than would likely be recommended as a mood enhancer, sleep aid, study aid, analgesic, or other treatment. Especially with children, you want to avoid risking overstimulation with the use of caffeine. However, caffeine, particularly when combined with theanine, has been found to offset sedative effects of a high dose of CBD and can also act as a study aid and mood enhancer. Alternatives to caffeine include theophylline, theobromine, or guarana, to name several.

5 mg of caffeine is believed to be a good dosage for a 50 mg CBD product, such as described in this example, because that is about the dosage of a typical serving of decaf coffee and 5 such doses (25 mg) would be a “weak coffee” option for children of practically any size. As a point of comparison, a typical cup of coffee (nowadays) has about 100 mg caffeine, which would mean a child would have to weigh 200 kg to be administered the equivalent of a single cup of coffee over the course of a day. Because inhibition of liver enzymes can improve with enterically delivered CBD, the caffeine can have a similarly delayed release so the CBD and caffeine become bioavailable at approximately the same time.

The name “theanine” without a prefix generally implies the enantiomer L-theanine, which is the form found in tea leaves and as a dietary supplement ingredient. It has been found that the combination of caffeine and theanine can improve word recognition, attention switching, mood, distractibility, relative to baseline. In a specific implementation, the ratio of caffeine to theanine is 1:2, which is believed to provide excellent correlation between dosage and the just-mentioned improvements. As such, in the example describe in the preceding paragraph, 10 mg of theanine is believed to be a good dosage for a 50 mg CBD product. To the extent caffeine has a delayed release, the theanine would also benefit from delayed release. Alternatives to theanine include perrottetinene (PET), THC, procaine, or the like.

Alternative liver enzyme inhibition products should be designed such that a patient receives 5-20 mg CBD per kg of body weight. In whatever manner this is delivered, caffeine and theanine, in an approximately 1:2 ratio, should be administered to counteract the sedative effects of CBD such that the patient does not receive more than is appropriate. For children, 100 mg caffeine per day, with 200 mg theanine, should have an advantageous impact on mood and concentration. For adults with some tolerance for caffeine, 250 mg in an energy drink dose may be desirable, though 250 mg over the course of a day would be more than enough for many adults (the equivalent of 2.5 cups of coffee). Because the goal of the product is liver enzyme inhibition and the primary purpose of caffeine and theanine is to ameliorate the sedative effects of CBD, it is unlikely desirable to exceed 250 mg caffeine through a treatment plan; adults can “self-medicate” with caffeinated drinks if they have the need for more.

An example of an edible toothpaste or gel product will now be discussed. The specific implementation described can be characterized as a morning toothpaste formulation with:
80 mg CBD
80 mg caffeine
160 mg theanine
10 mg (2%) peppermint oil
0.5 mg (0.1%) clove essential oil
0.5 mg (0.1%) fluoride
50 mg (10%) erythritol
1 mg (1%) sodium laurel sulfate (SLS)
50 mg (10%) carrageenan

The latter ingredients are best represented as a ratio of a nurdle. In a specific implementation, a nurdle of toothpaste is about 500 mg. In this specific implementation, 80 mg of CBD is provided in a dose. It may be desirable to increase nurdle size to 600 mg to make room for 100 mg CBD, 100 mg caffeine, and 200 mg theanine, which is believed to be an ideal dosage for a “strong cup of coffee” implementation as it matches the caffeine content of a modern cup of coffee and maintains a 1:1 ratio of CBD to caffeine and 1:2 ratio of caffeine to theanine. It is understood many individuals exceed the recommended nurdle size, which can be considered a waste of toothpaste and, in the case of a morning toothpaste, can result in over-indulgence of caffeine. Accordingly, it may be desirable to provide a dispenser that controls the quantity of toothpaste dispensed at a time to prevent unintentional overindulgence.

Potassium nitrate is believed to be a treatment for sensitive dentin due to nerve desensitization. While use of potassium nitrate in toothpaste for this purpose has increased every decade, the desensitization mechanism of potassium-containing toothpaste remains unclear. The use of nano-hydroxyapatite as an alternative to potassium nitrate has been increasing recently. It is believed an analgesic alternative can be as effective, particularly if supplemented with a chewing gum treatment. Methyl salicylate is frequently used in mouthwash as a localized analgesic. While methyl salicylate is used in some foods, such as breath mints, it is toxic in even relatively modest concentrations. Other salicylates, such as trolamine salicylate, are effective analgesics but are even more toxic.

CBD has dual-activity in that it addresses both pain and inflammation, which may be considered desirable in toothpaste. “Cannabidiol: From an Inactive Cannabinoid to a Drug with Wide Spectrum of Action,” by Zuardi (Brazilian Journal of Psychiatry, Vo. 30, No. 3, pp. 271-280, September 2008); “An Entourage Effect: Inactive Endogenous Fatty Acid Glycerol Esters Enhance 2-Arachidonoyl-Glycerol Cannabinoid Activity,” by Ben-Shabat et al. (European Journal of Pharmacology, Vol. 353, No. 1, pp. 23-31, July 1998); and “2-Arachidonoylglycerol (2-AG) Membrane Transport: History and Outlook,” by Hermann et al. (The AAPS Journal, Vol. 8, No. 2, pp. E409-E412, June 2006) are hereby incorporated by reference herein. Among other features, CBD binds to the CB₂ receptor on T-Cells, causing a reduction in inflammatory mediators, and binds to the transient receptor potential cation channel subfamily V member 1 (TrpV1), which is a pain receptor also known as the capsaicin receptor and the vanilloid receptor 1. Thus, CBD can be characterized as operating on the cell level to reduce both pain and inflammation.

In a specific implementation, microencapsulated CBD is provided as an analgesic in an edible toothpaste. In this specific implementation, 20 mg of the CBD is microencapsulated for buccal delivery to act as a localized analgesic in the mouth. CBD is degraded in the stomach but, as was discussed above, CBD is readily absorbed in mucosa. Although it is recognized not all patients will follow best practices, bioavailability estimates assume the amount of time spent brushing is 2 minutes, which is the time span over which the edible toothpaste remains in the mouth and which is long enough to make almost all of CBD formulated for buccal delivery bioavailable. Accordingly, when the product is used as directed, the CBD remains in the mouth long enough to achieve over 90% bioavailability. Thus, the amount of buccally delivered CBD can be added to that of enterically delivered CBD to determine a total systemic dose.

A low dose of CBD, of 50 mg or less, acts as a stimulant, making a 20 mg to 50 mg appropriate for formulations that have a goal of reducing the amount of CBD to below a dosage that would have a sedative effect. Because it is desirable to maintain a CBD-to-caffeine ratio of about 1:1, such a formulation could be characterized as a “weak cup of coffee” formulation. If a low dose of CBD is desired, the same dose of buccally delivered CBD as described above can be provided (20 mg in this specific implementation), but the amount of CBD formulated for enteric delivery can be reduced to 30 mg or eliminated entirely for a “decaf” option (though 20 mg of caffeine is still more than the caffeine content of a cup of decaffeinated coffee, which has about 5 mg of caffeine).

Because CB₂ receptors have a low affinity for CBD, large doses of CBD have increasingly advantageous pharmacological effects. However, CBD is relatively expensive, so the ratio of CBD to other components is more prone to cost/efficacy trade-offs than other components of the formulation. Alternatives can skimp on CBD and even drop the ratio of CBD-to-caffeine below the recommended 1:1 ratio, which should have acceptable effects so long as the caffeine is not in such a high dosage that it causes jitters.

A moderate dose of CBD, of 50 mg to 100 mg, can act as a sedative. While this may seem to act in cross-purposes to the stimulant effect desired in a product designed to, at least, improve wakefulness, it has been found the sedative effect reduces jitters, which are a side effect of stimulants like caffeine, particularly with overindulgence. This can address jitters for a formulation that includes caffeine or for a low-caffeine edible toothpaste supplemented with a caffeinated beverage. In addition to the 20 mg of CBD microencapsulated for buccal delivery in this specific implementation, 60 mg of the CBD is microencapsulated for enteric delivery. Accordingly, nearly 80 mg of CBD from the edible toothpaste should be bioavailable despite degradation of a small amount of the buccally delivered CBD in the stomach. In “full strength” alternatives, a nurdle of toothpaste recommended to be used by adults when brushing their teeth has more than 50 mg CBD but not more than 100 mg CBD.

A greater proportion of CBD may be desirable for the purpose of binding to theorized CBD3 receptors or other presently poorly defined receptors with less affinity for CBD. A high dose of CBD, over 100 mg up to less than 250 mg can have a sedative effect that may be effective for certain individuals, though it is unlikely desirable to reach 250 mg, which is approaching the liver enzyme inhibition dose. Although, currently marketed energy drinks have as much as 250 mg caffeine, it would be difficult to fit that much caffeine into a nurdle while maintaining a 1:1 ratio of CBD-to-caffeine (not to mention the 1:2 ratio of caffeine-to-theanine), so the upper ranges of CBD become largely irrelevant in this particular product due to volumetric constraints. If CBD is replaced with an endocannabinoid with (an estimated) 1,000 times the affinity of CBD, such as 2-Lineoleoyl-glycerol (3443-82-1) or 2-Palmitolyl-glycerol (23470-00-0), an alternative formulation could have 0.025 mg to 0.1 mg endocannabinoid, but the formulation would likely be much more expensive than CBD. An advantage of such a formulation is nurdle size can be kept small (around 500 mg) and about 40% more caffeine, assuming the caffeine-to-theanine ratio is kept at 1:2, can be fit into that volume.

An edible toothpaste can include an endocannabinoid mimetic, such as a synthetic cannabinoid, as a component. Examples of endocannabinoid mimetics are 18-Hydroxypalmitic acid (juniperic acid), AM404 (18718-77-6), and guineesine. In an alternative that includes, e.g., juniperic acid and CBD, the juniperic acid enables the CBD to linger for longer. Advantageously, because the “side effects” are reduced pain and inflammation, the composition would be safe as an oral treatment.

As was previously mentioned, it has been found that the combination of caffeine and theanine can improve word recognition, attention switching, mood, distractibility, relative to baseline. An effective ratio of caffeine to theanine is 1:2. An immediate effect of caffeine and theanine is not necessary, so an enteric delayed release is appropriate to maximize bioavailability of the components and to have a pharmacological effect at approximately the same time as the pharmacological effect of the CBD. Accordingly, in this specific implementation, 100% (80 mg) of the caffeine and 100% (160 mg) of the theanine is microencapsulated for enteric delivery.

In an alternative edible morning toothpaste, caffeine is omitted from the edible toothpaste and theanine is microencapsulated for (predicted) release when a morning coffee is consumed. While this has an appealing lifestyle approach that enables a person to brush in the morning and have their cup of coffee, it is more desirable from a therapeutic perspective to control the ratio of caffeine and theanine for optimal concentration enhancement effects. Nevertheless, benefits can be achieved for whatever product is demanded.

A foaming agent, thickener, dye, or flavoring can be added to a toothpaste for aesthetic purposes. However, some flavoring is also functional. For example, peppermint (essential) oil acts as a localized analgesic, antibacterial, and penetration enhancer in addition to providing a minty flavor. For a more immediate analgesic impact, CBD is combined with menthol (peppermint oil is about 40.7% menthol), which also binds to the TrpV1 receptor, but with an immediate response. It typically takes over 20 minutes for CBD absorbed through mucosa to provide significant pain relief but it lasts longer than the pain relief provided from menthol, making it a synergistic combination. As a penetration enhancer, the peppermint oil improves absorption of buccally delivered CBD, which can ameliorate the effects of patient error or haste when brushing, enabling patients to brush for less time than is recommended while still absorbing a large proportion of CBD orally. Although menthol is relatively toxic (a lethal dose can be as little as 50 mg/kg), it is frequently used as an additive to food in very low concentrations, so safe use of menthol in food is well-understood. (Methyl salicylate could also be used in a concentration of 0.04% concentration or less to be safe.) Camphor can be used in lieu of (or in addition to) menthol and is used in some foods and as a cough suppressant but may not be as effective an analgesic as menthol.

In the specific implementation described above, 10 mg (2%) of peppermint oil is provided in a dose. However, peppermint oil can have a pharmacological effect at much lower concentrations, as low as 0.02%. Although peppermint oil has a pharmacological effect at 2.5% or higher concentration, the menthol content of peppermint oil would make the product an over the counter (OTC) medication, which is less appealing for a mass-market toothpaste product. Microencapsulation of peppermint oil for buccal delivery can protect the peppermint oil from oxidation or other degradation during the manufacturing process, storage and transport, or after the product is opened.

Of varying degrees of toxicity, not all of which has been adequately researched, common antimicrobials used in oral hygiene products are chlorhexidine gluconate, benzethonium chloride, benzalkonium chloride, alkyldiaminoethylglycine hydrochloride, and chloroxylenol. Chloroxylenol is poisonous when ingested. Alkyldiaminoethylglycine hydrochloride is not approved as a food additive. Benzalkonium chloride is classed as a Category III antiseptic active ingredient by the United States Food and Drug Administration, which means “available data are insufficient to classify as safe and effective, and further testing is required”. Benzethonium chloride is not approved as a food additive in the U.S. or Europe, making it an unsuitable ingredient of an edible toothpaste. The Federal Drug Administration (FDA) in the USA recommendation is to limit the use of a chlorhexidine gluconate mouthwash to a maximum of six months and chlorhexidine gluconate is not approved as a food additive. Because the specific implementation described above is for a toothpaste, it is expected the unencapsulated essential oil will remain in the mouth for a relatively long time if used as directed, so a less aggressive antimicrobial than, e.g., chlorhexidine gluconate is believed to be adequate for routine oral antimicrobial treatments.

In a specific implementation, an edible toothpaste includes buccally delivered clove essential oil. Clove essential oil has a relatively pleasant taste and odor and clove essential oil has been found to be an effective anti-bacterial and anti-fungal substance and is a penetration enhancer. As a penetration enhancer, the clove essential oil improves absorption of buccally delivered CBD, which can ameliorate the effects of patient error or haste when brushing, enabling patients to brush for less time than is recommended while still absorbing a large proportion of CBD orally. Both in vivo and in vitro results have confirmed the efficacy of clove extract as an antimicrobial against Staphylococcus aureus, Escherichia coli, Brochothrix thermosphacta, Lactobacillus rhamnosus, Pseudomonas fluorescens, and other bacterial strains, molds, and yeasts. Alternatives include achillea species, basil, cardamom, cinnamon, coriander, cumin, fennel, galangal, garlic, ginger, marjoram, oregano, rosemary, thyme, and other spices that have been shown to have antibacterial and antifungal effects. Advantageously, these spices are routinely consumed in foods so safe usage is well understood.

Although its therapeutic effectiveness is disputed, particularly in South Korea and India, eugenol (clove essential oil is about 89% eugenol) is used to relieve toothache. In alternatives, other essential oils can be used instead of clove essential oil. Of note particularly in the toothpaste context, eugenol naturally occurs in several plants, including cloves, cinnamon, Cinnamomum tamala, nutmeg, sweet basil, African basil, holy basil, Japanese star anise, and lemon balm; eugenol can also be modified to taste like vanilla.

In the specific implementation described above, 0.5 mg (0.1%) clove essential oil is provided in a dose. However, clove essential oil can have a pharmacological effect at much lower concentrations, as low as 0.00001%. Due in part to the toxicity of clove essential oil, the highest concentration believed to be appropriate for an edible toothpaste product is 0.15%. Microencapsulation of clove essential oil for buccal delivery can protect the clove essential oil from oxidation or other degradation during the manufacturing process, storage and transport, or after the product is opened.

Although there are fluoride-free toothpaste alternatives, fluoride is generally seen as an important toothpaste ingredient. Fluoride is added to drinking water not only to prevent tooth decay, the effectiveness of which is now beyond dispute, but also to promote healthy bone growth. Consumption of fluoride in small amounts, therefore, is desirable.

In the specific implementation described above, 0.5 mg (0.1%) fluoride is provided in a dose. However, fluoride can have a pharmacological effect at much lower concentrations, as low as 0.0001%. It is also believed to be safe and effective in higher concentrations, as high as 1% being appropriate for an edible toothpaste product as described here.

Alcohol is used in mouthwash more frequently than toothpaste but sugar alcohols, including xylitol and sorbitol, are common in toothpaste and commercially available foods. Sugar alcohols do not cause tooth decay because they are not metabolized by oral bacteria. Sugar alcohols also do not dry out the mouth like alcohol, which is associated with halitosis and discomfort. Sugar alcohols can serve as a humectant in toothpaste. It has been found erythritol, another sugar alcohol, is more effective than xylitol and sorbitol in oral health care.

In the specific implementation described above, 50 mg (10%) erythritol is provided in a dose. It is generally undesirable to reduce the concentration of erythritol much below 10% but as low as 5% may still be adequate to provide a slightly sweet flavor and penetration enhancement, humectant, and thickening effect. A concentration as high as 30% can have beneficial effects but may not leave room for other components in the size of nurdle that is assumed in the specific implementation described here, though it is viable as a “weak cup of coffee” formulation. A concentration of up to 60% erythritol (and 39% water) is theoretically possible but such a concentration is not feasible without increasing nurdle size and crowding out other desirable components.

SLS is considered a generally recognized as safe (GRAS) ingredient for food use according to the USFDA (21 CFR 172.822). It is used as an emulsifying agent and whipping aid. It is used in toothpaste as a foaming agent and thickener. In this specific implementation, it is deemed appropriate for use in an edible toothpaste for essentially the same reasons it is used in commercially available (inedible) toothpastes. Because SLS is primarily aesthetic in its effects, though the function of allowing the product to be thick enough for placement on a brush is more than aesthetic, its concentration could be reduced or eliminated entirely. Commercially available toothpastes have concentrations of up to 2%, which is believed to be adequate as an upper limit, though there would be little harm (and little benefit) in increasing the concentration further.

Sugar alcohols, water, and carrageenan can all or in combination serve as a humectant in toothpaste. Carrageenan is also used as a thickener and stabilizer, which is useful for giving toothpaste a desired consistency, and a vegan alternative to gelatin. It is used in some commercially available toothpastes as a stabilizer. While its concentration could be reduced, it would likely be desirable to replace the carrageenan with some other thickener or stabilizer. A concentration as high as 30% can have beneficial effects but may not leave room for other components in the size of nurdle that is assumed in the specific implementation described here, though it is viable as a “weak cup of coffee” formulation. Alternatives to carrageenan include xanthan gum, cellulose, and glycerin.

Metronidazole, penicillin, amoxicillin, or some other antibacterial and potassium nitrate or some other component used to address pain, including sensitive teeth, can be microencapsulated for buccal delivery, as well.

Advantageously, microencapsulation of ingredients known to address oral hygiene issues provides an abrasive particle, a protective coating for bioactives, and a coating for releasing bioactives at target locations within the gastrointestinal tract. Calcium carbonate has some advantages for an edible toothpaste in that it has proven efficacy in toothpaste to provide antiseptic, teeth whitening, and plaque removal effect, plus it counteracts acidity in the mouth. Moreover, calcium carbonate and fluoride synergistically promote calcium replacement in teeth and calcium carbonate could serve as a calcium supplement (though the amount of calcium provided would be relatively low). In the specific implementation described above, calcium carbonate coats 20 mg of CBD, 100% of the caffeine, 100% of the theanine, 100% of the peppermint oil, and 100% of the clove essential oil for buccal delivery. Alternatives to calcium carbonate include magnesium carbonate, sodium bicarbonate, and various calcium hydrogen phosphates, to name a few.

The abrasive particles in an edible toothpaste should be relatively small, 4 to 12 μm, to provide an abrasive effect like that of commercially available toothpastes. The toothpaste should be abrasive enough to remove stains and plaque but not damage tooth enamel. The particle size could be increased to as high as 100 μm but that is a pretty grainy. Even at 50 μm, which is barely discernible when rubbed between the fingers, the toothpaste is pretty abrasive relative to commercially available toothpastes. At 1 μm, the particles may be too fine to have a desired abrasive effect, but this may be appropriate for a gentle edible toothpaste, such as a “magic” toothpaste, as described later, formulated for use after surgery or because the teeth or mouth are sensitive for some other reason. In the specific implementation described above, the calcium carbonate particles encapsulate bioactive components. In an alternative, a first subplurality of the calcium carbonate particles are microencapsulants and a second subplurality of the calcium carbonate particles are not microencapsulants.

Thus far, primarily microencapsulation for buccal delivery has been described in association with an edible toothpaste. In addition, shellac or an alternative material, examples of which are described above, can be used for enteric delivery, examples of which have been described above. In this specific implementation, all but 20 mg of CBD are enterically delivered, along with all the caffeine and theanine.

It may be desirable to provide a “magic” edible toothpaste that is formulated for a particular use. Magic mouthwashes are typically defined as prescription mouthwashes compounded in a pharmacy from a list of ingredients specified by a doctor. The term is expanded in this paper to apply to edible toothpaste and to include cosmeceuticals or edible toothpaste formulated to deliver fatty acids enterically. For example, a cognitive enhancing edible toothpaste can be formulated for morning use, a sleep aiding edible toothpaste for nighttime use, a cosmeceutical edible toothpaste formulated to treat a medical condition, or a pharmaceutical edible toothpaste compounded in a pharmacy from a list of ingredients specified by a doctor.

Because a patient will generally not use more toothpaste than fits on a toothbrush, macroscopic dosage can be controlled relatively easily and the person using the toothpaste can be relied upon to regulate intake as desired. Commercially available toothpaste dispensers generally dispense too much toothpaste, so it would be advantageous to control for dosage. Particularly when using essential oils, which are toxic when over-consumed, a controlled dosage dispenser may be desirable. The process of brushing one's teeth often includes two hands, a first hand to hold a tube of toothpaste and a second hand to open the tube of toothpaste and hold a brush while squeezing some toothpaste from the tube with the first hand. A hands-free controlled dosage dispenser can be advantageous when attempting to control dosage while one hand is holding a toothbrush.

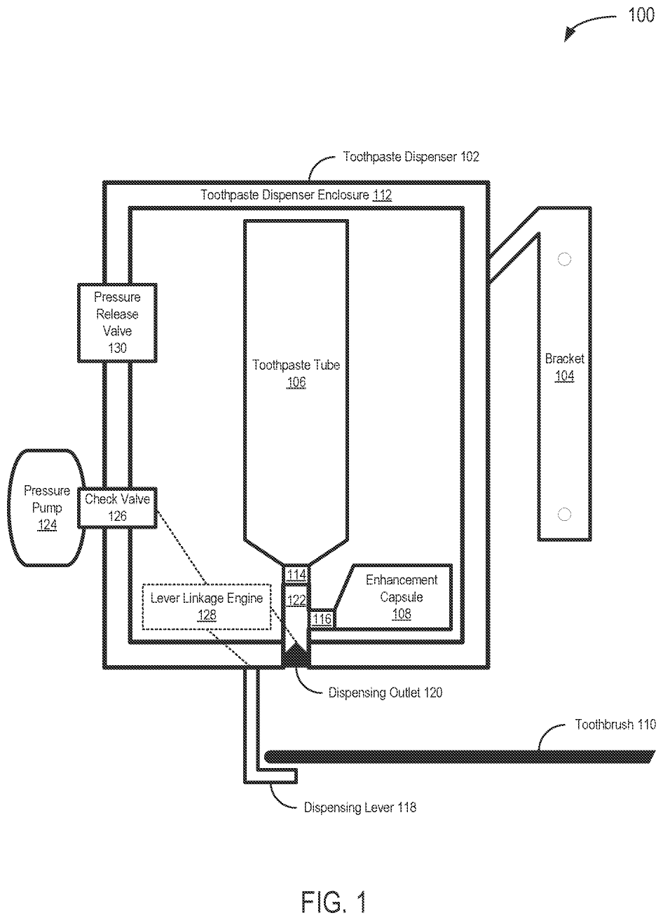

FIG. 1 is a diagram 100 of an example of a hands-free controlled dosage dispenser. The diagram 100 includes a toothpaste dispenser 102, a bracket 104, a toothpaste tube 106, a toothpaste enhancement capsule 108, and a toothbrush 110. The toothpaste dispenser 102 includes a toothpaste dispenser enclosure 112 operationally connected to the bracket 104; a toothpaste tube collar 114 operationally connected to the toothpaste tube 106; a toothpaste enhancement capsule collar 116 operationally connected to the toothpaste enhancement capsule 108; a dispensing lever 118 operationally connected to the toothpaste dispenser enclosure 112; a dispensing outlet 120 operationally connected to the toothpaste dispenser enclosure 112; a compounding chamber 122 adjoining the toothpaste tube collar 114, the toothpaste enhancement capsule collar 116, and the dispensing outlet 120; a pressure pump 124; a check valve 126 operationally connected to the pressure pump 126 and the toothpaste dispenser enclosure 112; a lever linkage engine 128 coupling the dispensing lever 122 to the dispensing outlet 120 and the dispensing lever 122 to the check valve 126; and a pressure release valve 130 operationally connected to the toothpaste dispenser enclosure 112.

In an expected use case, the toothpaste dispenser 102 is affixed to a wall (not shown) via the bracket 104. A patient or human or artificial agent thereof attaches the toothpaste tube 106 to the toothpaste tube collar 114. In a specific implementation, the toothpaste tube collar 114 is threaded to enable the patent or agent thereof to unscrew a cap (not shown) from the toothpaste tube 106 and engage the threads of the toothpaste tube 106 to the toothpaste tube collar 114. In an alternative, the toothpaste tube 106 is engaged with the toothpaste tube collar 114 with a latch, an adhesive, or in some other applicable manner. The patent or agent thereof also attaches the enhancement capsule 108 to the enhancement capsule collar 116. In a specific implementation, the enhancement capsule 108 is attached in the same manner as the toothpaste tube 106 is attached to the toothpaste tube collar 114. In the expected use case, the patient uses the toothbrush 110 to engage the dispensing lever 118 to move the dispensing outlet 120 to cause the compounding chamber 122 to release its contents onto the toothbrush 110.

The pressure pump 124 passes air through the check valve 126 into a pressurized chamber encompassed by the toothpaste dispenser enclosure 112, thereby increasing pressure within the pressurized chamber. The toothpaste tube 106 and the enhancement capsule 108, which are located within the pressurized chamber, are squeezed by the increased pressure to force respective contents of the toothpaste tube 106 and enhancement capsule 108 through the toothpaste tube collar 114 and the enhancement capsule collar 116, respectively, and into the compounding chamber 122. In an alternative to a pressurized chamber, the toothpaste tube 106 and enhancement capsule 108 can have their contents expelled using rollers, clamps, or some other applicable system.

The lever linkage engine 128 translates force from activation of the dispensing lever 118 to a force that moves the dispensing outlet 120, thereby causing the compounding chamber 122 to release its contents. In a specific implementation, the dispensing lever activates a crank pin and linkage to pull the dispensing outlet 120 into the compounding chamber 122, which causes the contents of the compounding chamber 122 to be squeezed out of the compounding chamber 122. When the dispensing lever 118 returns to its starting position, the lever linkage engine 128 activates the crank pin and linkage to allow a spring (not shown) to push the dispensing outlet 120 back into place, sealing the compounding chamber 122 from the outside environment. In an alternative, the lever linkage engine 128 operates to squeeze the compounding chamber 122 (e.g., by pushing walls of the compounding chamber 122 inward), flush the compounding chamber 122 with a fluid, or performs some other applicable technique to vacate the compounding chamber 122.

The lever linkage engine 128 also translates force from activation of the dispensing lever 118 to a force that causes the check valve 126 to allow intake of air from the pressure pump 124 into the pressurized chamber formed by walls of the toothpaste dispenser enclosure 112. In a specific implementation, the dispensing lever 118 directly engages and activates the pressure pump 124, which generates sufficient air pressure to cause the check valve 126 to open and allow the air to enter the pressurized chamber. In an alternative, the lever linkage engine 128 activates the pressure pump 124 with a crank pin and linkage operationally connected to the dispensing lever 118 or activates the pressure pump 124 in some other applicable manner. It may be necessary to “prime” the system by activating the dispensing lever 118 one or more times after a toothpaste tube 106 and/or enhancement capsule 108 has been newly attached to build up pressure within the pressurized chamber and to cause contents of the toothpaste tube 106 and/or enhancement capsule 108 to flow into the compounding chamber 122.

The pressure release valve 130 is intended to release air from the pressurized chamber if the pressure reaches a level that is deemed to be too high. The pressure release valve 130 can serve to reduce the risk of damaging the toothpaste dispenser 102 by causing too much pressure to build up within the pressurized chamber. In implementations that use something other than pressure, such as rollers, to expel contents from the toothpaste tube 106, the pressure release valve 130 may be eliminated.

The enhancement capsule 108 is intended to represent components that cause toothpaste that is in the toothpaste tube 106 to become “magic” toothpaste, which was described above. The enhancement capsule 108 is unnecessary if the toothpaste in the toothpaste tube 106 already has all desired components included in its formulation. In an alternative that does not include the compounding chamber 122, contents of the enhancement capsule 108 can be released into a first chamber and contents of the toothpaste tube 106 can be released into a second chamber, and the combined contents can be compounded on the brush when both are released by the dispensing outlet 120.

An example of another edible toothpaste or gel product will now be discussed. The specific implementation described can be characterized as a nighttime toothpaste formulation with:
100 mg CBD
200 mg theanine
2 mg pyridoxine tripalmitate
10 mg (2%) peppermint oil
0.5 mg (0.1%) clove essential oil
20 mg (4%) potassium nitrate
0.5 mg (0.1%) fluoride
50 mg (10%) erythritol
1 mg (1%) sodium laurel sulfate (SLS)
50 mg (10%) carrageenan

In a specific implementation, a nurdle of edible nighttime toothpaste includes 50 mg CBD microencapsulated for buccal delivery. This moderate dose of CBD should have a relatively fast-acting mild sedative effect on some patients that is not counter balanced with a stimulant like caffeine, and when the remainder of the CBD, which is enterically delivered, becomes bioavailable, the sedative effect should be more pronounced.

Theanine, as noted above, improves cognition synergistically with caffeine. For a nighttime toothpaste formulation, however, it is undesirable to stimulate the patient. Nevertheless, theanine can be used as a mood enhancer without the synergistic effects of caffeine.

The desired ratio of pyridoxine tripalmitate to theanine is between 1:50 and 1:100. Alcoholism, liver disease, and some drugs can cause low levels of B6, making a higher dose for certain patients useful. It should also be noted that 100 mg is the maximum daily allowance for B6 (30 mg for a 1 year old), making a dosage below 30 mg desirable, with 3 mg being a safe option for small tube of toothpaste (of about 10 doses) because, even if consumed by a child, it would be safe. The risk can be ameliorated with a dosage control pump and child safety lock.

An accurate dosage of B6 may be advantageous when addressing sensitive issues, such as controlling intake of folate, B6, and B12, during pregnancy. In women's health alternative implementation of the product provided in this example, issues associated with post-menopausal syndrome (PMS) can be addressed by adding magnesium in an approximately 4:1 ratio magnesium to pyridoxine triacetate. In an alternative, the formulation provided above is modified to include 8 mg magnesium, while maintaining the indicated ratios of essential oils, fluoride, erythritol, SLS, and carrageenan, which may or may not modestly increase nurdle size.

Peppermint oil and clove essential oil are intended to provide the same benefits described above with reference to a daytime edible toothpaste formulation.

CBD can be used instead of potassium nitrate or a salicylate to treat pain. However, in this specific implementation, a sensitive dentin formulation includes potassium nitrate for buccal delivery. Although commercially available toothpastes include 5% potassium nitrate, it is not clear such a high concentration, while safe, is better than a 4% concentration at reducing dentin sensitivity. Moreover, a regimen of daily doses of CBD, as would be provided in a daily-use toothpaste, are expected to make a 3% concentration viable, though this specific implementation includes more conservative 4% concentration. If it is determined a lower concentration is just as effective as a 4% concentration, the concentration of potassium nitrate can be reduced.

In an alternative, the potassium nitrate is removed or replaced with some other component, such as water-soluble peptide extracts formulated for enteric delivery. Commercially available probiotic drinks have theoretical health benefits from probiotics, but probiotics are extremely prone to degradation in the stomach; probiotics are best delivered enterically. In a specific implementation, water-soluble peptide extracts are microencapsulated for enteric delivered. Relative to the daytime edible toothpaste formulation described previously, the elimination of caffeine in the formulation leaves room for a water-soluble peptide extract without increasing nurdle size; similar nurdle size may be desirable when pairing daytime and nighttime edible toothpaste in a comprehensive oral care product suite. Microencapsulated water-soluble peptide extracts could also be included in a “weak coffee” formulation of a daytime edible toothpaste or nurdle size could be increased to 600 mg (or more) while maintaining approximately the same concentrations of peppermint oil, clove essential oil, fluoride, erythritol, SLS, and carrageenan to make room for the water-soluble peptide extracts.

Fluoride is intended to provide the same benefits described above with reference to a daytime edible toothpaste formulation but may also improve dentin sensitivity reduction when paired with potassium nitrate. Erythritol, SLS, and carrageenan are intended to provide the same benefits described above with reference to a daytime edible toothpaste.

This specific implementation can include components to treat a medical condition without impacting nurdle size relative to a daytime edible toothpaste's nurdle size by removing caffeine from the formulation, leaving room for other components. As was mentioned above with reference to the inclusion of potassium nitrate in the formulation, relative to the daytime edible toothpaste formulation described previously, the elimination of caffeine in the formulation leaves room for another component (a water-soluble peptide extract was the example provided above) without increasing nurdle size. Advantageously, similar nurdle size can be maintained when providing a “magic” nighttime edible toothpaste allowing a patient to follow the same oral care regimen including a daytime edible toothpaste, with the same amount of toothpaste, as they did prior to being provided with the magic nighttime edible toothpaste.

Mouthwash may include denatured alcohol, such as methyl alcohol, which should not be consumed because it can cause blindness, organ failure, and death. Other ingredients of mouthwash that are harmful if over-consumed include chlorhexidine gluconate, ethyl alcohol, hydrogen peroxide, and methyl salicylate. Minor and transient side effects of mouthwashes are common, such as taste disturbance, tooth staining, sensation of a dry mouth, etc. Alcohol-containing mouthwashes may make dry mouth and halitosis worse because alcohol dries out the mouth. Alcohol is added to mouthwash not to destroy bacteria (although it can) but to act as a carrier agent for essential active ingredients such as menthol, eucalyptol and thymol which help to penetrate plaque; it may also be provided in relatively high proportion so the mouthwash has a “bite” to it.

Rinsing with water or mouthwash after brushing with a fluoride toothpaste can reduce the availability of salivary fluoride. This can lower the anti-cavity re-mineralization and antibacterial effects of fluoride. Fluoridated mouthwash may mitigate this effect or in high concentrations increase available fluoride.

An example of a drinkable mouthwash will now be discussed. The specific implementation described can be characterized as a morning mouthwash formulation with:
100 mg CBD
100 mg caffeine
200 mg theanine
200 mg (1%) peppermint oil
10 mg (0.05%) clove essential oil
10 mg (0.05%) fluoride
2 g (10%) erythritol
40 mg (1%) sodium laurel sulfate (SLS)

The formulation provided in this specific implementation is similar to the formulation for an edible morning toothpaste, described previously, but the expected dose of a mouthwash is 40 times more (20 grams) than that of a nurdle of toothpaste (500 mg). Thus, there will be far more water mixed with the formulation unless the mouthwash is provided in powdered or tablet form, in which case water can be added later.

A toothpaste can be kept in an opaque tube to prevent oxidation and the toothpaste itself is typically opaque and insulative, protecting bioactives within the paste so long as the paste has properties that do not degrade the bioactives and/or the bioactives are encapsulated to protect them from chemically reacting with the paste. Mouthwashes are frequently sold in transparent containers, which may be more appealing to a consumer. This makes microencapsulation even more important in mouthwash than in toothpaste due to the risk of photooxidation. Bottles of mouthwash are also more prone to expose contents to air than tubes of toothpaste, further increasing oxidation risk through contact with air.

As with the edible toothpaste described above, a first portion of the CBD is formulated for buccal delivery and a second portion of the CBD is formulated for enteric delivery. In this specific implementation, 20 mg of the CBD is microencapsulated for buccal delivery to act as a localized analgesic in the mouth. CBD is degraded in the stomach but, as was discussed above, CBD is readily absorbed in mucosa. Although it is recognized not all patients will follow best practices, bioavailability estimates assume the amount of time mouthwash is kept in the mouth is 30 seconds to one minute, which is long enough to make almost all of CBD formulated for buccal delivery bioavailable. Accordingly, when the product is used as directed, the CBD remains in the mouth long enough to achieve over 90% bioavailability. Thus, the amount of buccally delivered CBD can be added to that of enterically delivered CBD to determine a total systemic dose.

The technique for buccal delivery for a mouthwash may be different from that of a mouthwash. Specifically, calcium carbonate may not dissolve adequately to release contents coated therewith. To address this issue, the buccal coating can be temperature sensitive. For example, shea butter, cocoa butter, or hemp seed oil could be used to coat CBD and, when the mouthwash approaches body temperature, the butter melts, releasing the CBD. For additional photooxidation protection, zinc oxide (which is on the GRAS list) or some other material that reflects or absorbs harmful electromagnetic frequencies can be mixed into the butter to provide broad spectrum light absorbance. In this specific implementation, the particles are 50 microns or smaller.

A portion of the CBD and the caffeine and theanine are formulated for enteric delivery as described above with reference to the edible toothpaste. The peppermint oil and clove essential oil are formulated for buccal delivery as described above.

The specific implementation is fluoridated to be valuable immediately after brushing. Although it is best practices to wait 30 minutes or even as much as 2 hours after brushing before rinsing with anything, many people do not follow these best practices. In an alternative, fluoride concentration is reduced to that of drinking water (0.7 mg/L) and instructions to wait 30 minutes to 2 hours after brushing (and to not rinse with water after brushing) can be omitted.

In a specific implementation, an example of a drinkable mouthwash is an antiseptic, analgesic, anti-inflammatory, anti-fungal suspension that acts as a saliva substitute to neutralize acid and keep the mouth moist in xerostomia (dry mouth); the drinkable mouthwash also controls bad breath by leaving the mouth with a pleasant taste. In a specific implementation, erythritol is used instead of alcohol at least in part because it does not dry the mouth and in part because it prevents tooth decay.

Magic mouthwashes are used prior to and after oral surgery procedures such as tooth extraction or to treat the pain associated with mucositis caused by radiation therapy or chemotherapy. They are also prescribed for aphthous ulcers, other oral ulcers, and other mouth pain. Magic mouthwashes are prescription mouthwashes compounded in a pharmacy from a list of ingredients specified by a doctor. Despite a lack of evidence that prescription mouthwashes are more effective in decreasing the pain of oral lesions, many patients and prescribers continue to use them. There has been only one controlled study to evaluate the efficacy of magic mouthwash; it shows no difference in efficacy among the most common formulation and commercial mouthwashes such as chlorhexidine or a saline/baking soda solution. Current guidelines suggest that saline solution is just as effective as magic mouthwash in pain relief or shortening of healing time of oral mucositis from cancer therapies. Moreover, mouthwashes that are spit out can be harmful to the environment. For example, antibiotics spit into a drain can make bacteria more resistant through natural selection.

Saline and baking soda mouthwashes are generally not swallowed and adding salt or sodium bicarbonate to a drinkable mouthwash in the concentrations necessary to achieve the effects of saline and baking soda mouthwash or mouth bath is challenging. However, a drinkable magic mouthwash that is used after a saline mouthwash or mouth bath can enhance oral hygiene and pain control. A drinkable magic mouthwash that is used as a mouth bath before a saline mouthwash can be used for its pain management properties to ameliorate some of the discomfort of a saline mouthwash.

A specific implementation can be characterized as a nighttime mouthwash formulation with:
100 mg CBD
200 mg theanine
2 mg pyridoxine tripalmitate
200 mg (1%) peppermint oil
10 mg (0.05%) clove essential oil
300 mg (3%) potassium nitrate
10 mg (0.05%) fluoride
2 g (10%) erythritol
40 mg (1%) sodium laurel sulfate (SLS)

A portion of the CBD and the theanine are formulated for enteric delivery as described above with reference to the edible toothpaste. In other respects, the components are used for purposes like those described above with reference to an edible nighttime toothpaste formulation.

For most food and drink, whether the product is presented in a clear bottle or a can, is presented in a salt shaker that has an opening at the top that exposes the product to oxygen, is an additive to a food item that is going to be cooked before it is served, or is subjected to other oxidation risks is more likely than would be experienced by a toothpaste or a mouthwash. Drinkable mouthwashes, which can be sold in a manner similar to breath strips or breath mints, may be even more prone to packaging that is appealing to a consumer market but is less protective of bioactives susceptible to oxidation.

Interactions between contents of a food or beverage, including carbonation, flavoring, packaging, serving temperature, and the like can impact bioactives in the food or beverage. Infusing a liquid with nitrogen instead of carbon dioxide, while more expensive, can avoid increasing acidity, as is the case with carbonation. Microencapsulation with IR opaque materials (insulation) can limit degradation from temperature. To the extent a drink is provided in a transparent or translucent container, coloring the microencapsulation material can retard photooxidation. UV opaque encapsulants can also be used, as can reflective materials, potentially with aesthetically desirable effects like sparkling in addition to the functional effect of reflecting light to retard photooxidation. Thus, advantageously, microencapsulation can be used to retard oxidation prior to consumption throughout the supply chain, from manufacturing to storage and finally to consumption.

Microencapsulation is frequently imperfect, particularly when the particles approach 1 micron in diameter, making it desirable to include pigments that can protect components that are imperfectly coated. Colors in fluid in which the bioactive is free floating can protect against photooxidation if the color absorbs or reflects in the relevant frequency bands. Such coloration can also be added to encapsulants. For CBD, the encapsulant should absorb or reflect light in the frequency ranges of violet visible light (specifically in the case of CBD, 375-420 nm) and red visible light (specifically in the case of CBD, 650-740 nm). It is advantageous to absorb light of other frequencies for other bioactives.

In a specific implementation, an energy drink includes microencapsulated fatty acids. As was previously mentioned, caffeine combined with theanine has advantageous stimulant and cognitive improvement effects, and CBD can have a sedative effect in moderate doses (approximately 100 mg). Energy drinks can have as high as 250 mg caffeine content, but it is believed the sedative (anti-jitter) effect of CBD is not or is only modestly improved with a higher dose. Accordingly, an energy drink formulation can include 100 mg CBD, 250 mg caffeine, and 500 mg theanine. CBD is more degraded in the stomach than caffeine, but both can benefit from enteric delivery and the ratio of caffeine to theanine can be tightly controlled to ensure bioavailability in the small intestine.

Some foods have been shown to have beneficial effect at specific locations within the human body. For example, water-soluble peptide extracts, bacterial peptides released from dairy products like yogurt, have antimicrobial properties and have been shown to inhibit proliferation of, e.g., HT29 human colon cancer cells. Cranberry juice has been argued to be effective against urinary tract infections but in a 2015 study cranberry capsules lowered the risk of UTIs by 50% in women who had catheters in place while undergoing gynecological surgery, with implications related to delayed release, high bioactive concentration, and the like. By microencapsulating the foods that are best made bioavailable via enteric delivery, degradation in the stomach can be avoided.

A specific implementation of an energy shot formulation is:
100 mg CBD
250 mg caffeine
500 mg theanine
300 mg (0.5%) peppermint oil
6 mg (0.01%) fluoride
12 g (20%) sorbitol

Energy drinks come in many sizes, two common ones of which are an energy shot, which is less than 60 grams (though 60 grams is assumed for this specific implementation), and a 16 oz. can, which is about 448 grams. The energy shot is about 3× the daytime mouthwash implementation dose described above and a similar formulation is used in this specific implementation. However, the essential oils and fluoride are reduced in concentration or eliminated and erythritol is replaced with a less expensive alternative, such as sorbitol, because the product is not sold as a cavity preventive drink.

Some substances, such as tobacco, cloves, marijuana, and some medicines, are consumed through inhalation, which may include smoking, vaping, or aerosol delivery. The deleterious effects of smoking have been well-documented and there is increasingly alarming evidence vaping may be even worse in certain situations. However, consumption of a substance through smoking provides a different degree of impact from the various components of the substance than eating or drinking the substance. Matching the effect of a substance that is smoked with a substance that is drunk is referred to as achieving the entourage effect in this paper.

A specific implementation of an orally delivered formulation with an entourage effect is as described above for an energy shot, but with the addition of 100 mg PET (a legal alternative to THC). CBD and PET can be formulated for enteric delivery to achieve the entourage effect in the proportion that is desired. To the extent it is desirable to have a different ratio, such as a 2:1 CBD-to-PET ratio, the desired ratio can be used. This ratio selection would mimic the menus found in legal pot dispensaries when explaining the components of an available plant product. An energy shot that is formulated to provide a combination of pharmacological effects that would be expected if inhaling the CBD and PET can be characterized as an entourage energy shot (or energy shot that delivers an entourage effect).

A formulation can include an immediate (buccal) release of CBD and PET in a 4:1 (or 4× upscaling of CBD if a different entourage ratio was intended) ratio because of the reduced bioavailability of CBD taken orally (but note if the CBD is kept in the mouth longer, such as is the case when using brushing teeth or using mouthwash, the ratio can be flattened by an amount that depends upon the amount of CBD delivered buccally). Thus, as an alternative to this specific implementation, 200 mg CBD is formulated for buccal delivery, 50 mg CBD is formulated for enteric delivery, 50 mg PET is formulated for buccal delivery, and 50 mg PET is formulated for enteric delivery. Such a formulation can be characterized as a fast-acting entourage energy shot (or a fast-acting energy shot that delivers an entourage effect). In an alternative, the 500 mg of theanine are removed from the formulation.

Enterically delivered fatty acids can be designed for an hourly delayed release for up to approximately 8 hours. If an even longer staged release is desired, pegylation can be used to slow the metabolization of fatty acids to maintain an appropriate ratio of the fatty acids in the bloodstream. The half-life of CBD in the bloodstream is about 1.3 days for an infrequent user and up to 10× that amount (5-13 days) for a frequent user. This is likely due to enzyme inhibition, though an explanation of the process may be better understood in the future. It may be undesirable to cause CBD to remain in the bloodstream for significantly longer than would be expected. Accordingly, a delayed release of greater than 8 hours may only be desirable for medicinal applications, such as an epilepsy drug supplement.

A 16 oz. can formulation is envisioned to have more water but not more CBD, PET, caffeine, theanine, or fluoride, and roughly the same concentration of peppermint oil and sorbitol as the energy shot formulation described above. A hybrid “swish and swallow” energy shot is envisioned with components like those of the morning mouthwash described above, but with twice as much caffeine and theanine, and in approximately the same serving size as a mouthwash dose.

A specific implementation of an entourage shot formulation is:
100 mg CBD
100 mg PET
100 mg caffeine
200 mg theanine
300 mg (0.5%) peppermint oil
6 mg (0.01%) fluoride
12 g (20%) sorbitol

The entourage shot retains some caffeine to counteract the sedative effect of CBD. In a formulation that upscales CBD, it is likely undesirable to upscale the caffeine and theanine. However, for a formulation that downscales CBD for a desired entourage effect, CBD and caffeine should maintain a 1:1 ratio and caffeine and theanine should maintain a 1:2 ratio unless a “strong cup of coffee” entourage shot is desired.

A drink additive is envisioned for producing a desired entourage effect. In this specific implementation, a pint (about 473 grams) of caffeinated fluid is supplemented with an entourage packet. A specific implementation of an applicable caffeinated fluid formulation is:
100 mg CBD
100 mg caffeine
200 mg theanine
473 mg (0.1%) peppermint oil
94.6 g (20%) sugar

The caffeinated beverage in this example is the equivalent of a strong cup of coffee, though with a lower concentration of caffeine, and is rather sweet. Weak coffee alternatives would downscale CBD along with caffeine, maintaining the 1:1 ratio between CBD and caffeine and the 1:2 ratio between caffeine and theanine. (A decaf option could downscale all the way to 5 mg CBD, 5 mg caffeine, and 10 mg theanine or eliminate the components entirely.)

A low-calorie variant can replace the sugar with sorbitol on a 1:1 basis and you can add 3.5 mg (approximately 7 mg/L) fluoride, which is about the same concentration of fluoride as is found in drinking water. You can also add components as described above for mouthwash, though likely in lower concentrations, for a swish and swallow formulation.

A specific implementation of an entourage packet for addition to the caffeinated fluid has a formulation of:
100 mg CBD
100 mg PET

When a formulation is for a food or beverage additive that is sprinkled on the food or in the drink, buccal delivery can be accomplished with, for example, trehalose. CBD and PET can be kept stable in trehalose and released when the trehalose is dissolved in water. In a specific implementation, the entourage packet contents are formulated as a powder using a network sugar such as trehalose. Alternatives to PET include the same alternatives as described above for theanine.

Chocolate shields fatty acids, such as CBD, which can help the fatty acids, should they make it to the stomach, survive until they reach the small intestines. Advantageously, imperfect buccal delivery can be augmented with chocolate (indeed, the chocolate, given it includes cocoa butter, can be used as a buccal coating for a bioactive). A chocolate powder version coats the CBD and PET formulated for buccal delivery in chocolate.

1. A liver enzyme inhibitor medication comprising:
50 mg to 200 mg CBD per 10 kg of patient body weight per daily dosage, wherein 12.5 mg of the CBD is encapsulated for buccal delivery;
0.5 mg to 5 mg of pyridoxine tripalmitate;
a dual-functional sweetener and penetration enhancer;
a stimulant;
theanine.

2. The liver enzyme inhibitor medication of claim 1 wherein the dual-functional sweetener and penetration enhancer includes 50 mg xylitol.

3. The liver enzyme inhibitor medication of claim 1 wherein the stimulant includes 5 mg caffeine per 10 kg of patient body weight and a ratio of 1:2 caffeine:theanine.
